(19) United States
(12) Patent Application Publication
(10) Pub. No.: US 2023/0151107 A1
(43) Pub. Date: May 18, 2023

(54) ANTI-CD6 ANTIBODY COMPOSITIONS AND METHODS FOR TREATING AND REDUCING NEGATIVE EFFECTS OF A CORONAVIRUS INCLUDING COVID-19

(71) Applicants: BIOCON LIMITED, Bangalore (IN); CENTRO DE INMUNOLOGÍA MOLECULAR, La Habana (CU)

(72) Inventors: Pradip NAIR, Bangalore (IN); Jose Enrique Montero CASIMIRO, Carlsbad, CA (US); Kiran MAZUMDARSHAW, Bangalore (IN); Usha BUGHANI, Bangalore (IN); Sandeep Nilkanth ATHALYE, Bangalore (IN); Melarkode Subbaraman RAMAKRISHNAN, Bangalore (IN); Tania CROMBET RAMOS, La Habana (CU); Kalet LEÓN MONZÓN, La Habana (CU); Mayra RAMOS SUZARTE, La Habana (CU)

(21) Appl. No.: 17/916,910

(22) PCT Filed: Mar. 3, 2021
(86) PCT No.: PCT/IB2021/052793

§ 371 Date: Oct. 4, 2022

(30) Foreign Application Priority Data
Apr. 4, 2020 (IN) 202041014994
Apr. 17, 2020 (CU) CU-2020-0027

(51) Int. Cl. C07K 16/28; A61K 39/395; A61K 45/06; A61P 31/14

(52) U.S. Cl. CPC C07K 16/2896; A61K 39/3955; A61K 45/06; A61P 31/14; A61K 2039/545

(57) ABSTRACT

The present invention provides the use of anti-CD6 antibodies that specifically bind to domain 1 of CD6 for treating effects of a coronavirus or bacterial agent and particularly COVID-19 and variants thereof. The anti-CD6 antibodies of the present invention exhibit therapeutic activity by reducing the overactive immune response, such as the high expression levels of cytokines.

### CROSS-REFERENCE TO RELATED APPLICATIONS

This application claims the priority of Indian Provisional Application No. 202041014994 filed on Apr. 4, 2020; and Cuban Provisional Application No. 2020-0027 filed on Apr. 17, 2020, which are hereby incorporated by reference in its entirety.

### FIELD OF INVENTION

The present invention is directed to anti-CD6 antibodies, including non-depleting anti-CD6 antibodies, methods of use and compositions for treating and reducing the negative effects caused by infection agents such as bacterial and viruses particularly the coronaviruses, wherein the negative effects include the triggering of a cytokine storm or a cytokine release syndrome.

### BACKGROUND OF INVENTION

More than 100 years after the last pandemic (1918), the world today is in the midst of another pandemic caused by an RNA virus which the International Committee on Taxonomy of Viruses (ICTV) has named ‘severe acute respiratory syndrome coronavirus 2 (SARS-CoV-2) and disease caused is called the COVID-19. The virus belongs to the family of coronaviruses and has as of today, spread to all continents and is therefore expected to stay for a long time.

Today over 115 million people worldwide are infected with the virus across the world. While many have recovered, about 15% of the confirmed cases progress to the severe phase, with a higher chance for patients over 65 years of age to progress into the severe phase. Lethality is around 3% from data across the world.

The virus is primarily transmitted by respiratory droplets from an infected patient that enter the nasal, oral passage or eyes of another person. It takes around 2 to 14 days to develop symptoms in an infected patient, with an average of 5 days. The three coronaviruses (severe acute respiratory syndrome coronavirus (SARS-CoV), Middle East respiratory syndrome coronavirus (MERS-CoV) and SARS-CoV-2 are transmitted via respiratory droplets and can replicate in the lower respiratory tract and cause pneumonia.

The World Health Organization (WHO) has reported that almost 80% of the cases have mild to moderate infection (Classified as cough and fever but not requiring hospitalization) 13.8% have severe and 6.1% have critical disease. The median incubation period of the infection is about 4-5 days before symptom onset, with 97.5% of symptomatic patients developing symptoms within 11.5 days. At hospitalization, patients with COVID-19 exhibit a fever, dry cough and sometimes dyspnoea. Within 5-6 days of symptom onset, SARS-CoV-2 viral load reaches its peak and in severe cases the infection progresses to acute respiratory distress syndrome (ARDS), around 8-9 days after symptom onset.

Recent data suggests that Human pathogenic coronaviruses (severe acute respiratory syndrome coronavirus [SARS-CoV] and SARSCoV-2 (COVID-19) bind to their target cells through angiotensin-converting enzyme 2 (ACE2), which is expressed by epithelial cells of the lung (Type II, alveolar cells) intestine, kidney, and blood vessels. The expression of ACE2 is substantially increased in patients with type 1 or type 2 diabetes and hypertension who are treated with ACE inhibitors and angiotensin II type-I receptor blockers (ARBs). This likely explains why patients above age 65 who are usually associated with these co morbidities are more susceptible to this disease. These patients exhibit symptoms similar to the SARS (severe acute respiratory symptom) outbreak in 2002-2003 such as mild, cold-like symptoms, including fever, cough, fatigue, shortness of breath, muscle aches, and loss of taste and/or smell. SARS-CoV-2 infection triggers a pro-inflammatory response in some patients that includes high levels of cytokines, including IL-6, fibroblast growth factor (FGF), granulocyte macrophage colony stimulating factor (GM-CSF), tumor necrosis factor and vascular endothelial growth factor. IL-6 levels have been found to be directly correlated with increased mortality and inversely correlated with lymphocyte count, suggesting cytokine release syndrome may impede the adaptive immune response (Li, et al. “Coronavirus infections and immune responses” J 322 Med Virol. 2020 April; 92(4):42 4-32).

Once the lung is infected, the virus uses RNA polymerase to replicate in the alveolar cells and use the cellular machinery to translate the RNA to proteins. Finally, multiple viral particles are produced and released. This causes reinfection and the viruses can be transmitted to other people.

There are no standard treatment options for COVID19 infections caused by this new virus. However, multiple drugs are being tried in combination based on the virus infection pathway. Other drugs in development or currently being tested include multiple companies focusing on vaccines and monoclonal antibody therapy. Actemra from Roche (Anti IL6) is in clinical trials for COVID, this drug has been repurposed for this indication from autoimmune disease. Kevzara from Sanofi also is an antibody blocking IL6 and is in clinical trials. Regeneron is developing antibodies to viral spike proteins hoping to prevent its interaction with ACE2 receptor. Takeda is developing Hyper immunoglobulin from patients who responded, for passive immunity. CytoSorb therapy is based on an extracorporeal blood purification procedure that can effectively reduce excessive levels of inflammatory mediators in critically ill patients.

The need for new and more effective therapeutics to treat the negative effect of COVID19 continues to increase, particularly those targeting emerging and re-emerging infectious diseases and pathogens. As such, there is a necessity to provide a new and effective treatment to reduce the negative effects of COVID-19 and variants thereof.

### SUMMARY OF THE INVENTION

The present invention provides the use of an anti-CD6 antibody, including a non-depleting anti-CD6 antibody that specifically binds to domain 1 of CD6 for treating and reducing the effects of a coronavirus and particularly Novel Coronavirus (COVID-19) and variants thereof. Particularly, the anti-CD6 antibody of the present invention exhibits therapeutic activity by reducing the overactive immune response, such as the high expression levels of cytokines.

The present invention provides the use of an anti-CD6 antibody, including a non-depleting anti-CD6 antibody that specifically binds to domain 1 of CD6 for treating and reducing the effects of a bacterial infection agent and particularly group A streptococci and pneumococci. Particularly, the anti-CD6 antibody of the present invention exhibits therapeutic activity by reducing the overactive immune response, such as the high expression levels of cytokines.

Accumulating evidence suggests that some patients with severe COVID-19 are experiencing a cytokine storm syndrome. Cytokines are inflammatory immunologic proteins that are there to fight off infections and ward off cancers but too many can result in an immune system gone wild. The body's own killer immune cells, in such an increased amount, can lead to organ failure and death. Currently, respiratory failure from acute respiratory distress syndrome (ARDS) is the leading cause of mortality of patients with COVID-19. Thus, the present invention provides for a novel approach to reduce inflammatory cytokine proteins, which can include interleukins IL-1, IL-6 and IL-2, and interferon-gamma and to treat ill individuals with complications of hyper-inflammation due to COVID-19 and variants thereof.

In one aspect, the present invention also provides a method of treating a coronavirus-infected cell or organism comprising treatment with a therapeutically effective amount of an anti-CD6 antibody comprising heavy and light chain variable regions comprising amino acid sequences as set forth in SEQ ID NOs: 1 (variable region of heavy chain) and 2 (variable region of light chain) or an amino acid sequence having at least 98% identity thereto comprising SEQ ID Nos: 1 (variable region of heavy chain) and 3 (variable region of light chain) according to the present invention.

In another aspect, the present invention also provides a method of treating a coronavirus-infected cell or organism comprising treatment with a therapeutically effective amount of an anti-CD6 antibody having heavy and light chain regions comprising or consisting of amino acid sequences as set forth in SEQ ID NOs: 4 (heavy chain) and 5 (light chain) or an amino acid sequence having at least 98% identity thereto according to the present invention. An anti-CD6 antibody comprising or consisting of SEQ ID NO: 4 and 6 is included in the present invention.

The antibody is administered to patients intravenously, in a dose range between about 0.5 mg/kg of body weight and about 8 mg/Kg of body weight. Doses between 1 mg/kg and 6 mg/kg are preferable. The antibody is preferably administered at least twice to the patient up to a maximum of 5 doses. The time elapsed between two consecutive administrations will be between 24 and 96 hours. The preferred administration schedule is two to three doses spaced 72 hours apart.

In another aspect, the present invention provides a method for alleviating the cytopathic destructive effects of COVID-19 infection and variants thereof in a human patient comprising administering to said patient a therapeutic amount of an anti-CD6 antibody comprising SEQ ID NO: 1 (variable region of heavy chain) and SEQ ID NO: 2 (variable region of light chain) or 3 (variable region of light chain) according to the present invention.

In yet another aspect, the present invention provides for therapeutic compositions comprising a therapeutic amount of an anti-CD6 antibody comprising amino acid sequences of SEQ ID NOs: 4 (heavy chain) and 5 or 6 (light chain) according to the present invention in a pharmaceutically acceptable carrier.

In addition, because the use of more than one active agent may provide a better therapeutic composition, and this is particularly true when the different agents act by different mechanisms, this invention also includes therapeutic compositions including both an anti-CD6 antibody comprising amino acid sequences SEQ ID NO: 4 (heavy chain) and SEQ ID NO: 5 or 6 (light chain) according to the present invention together with one or more other antiviral agents, such as Reverse transcriptase (RT) inhibitors—interfere with a critical step during the virus life cycle and keep the virus from making copies of itself; Nucleoside analogs—nucleoside analogue to inhibit coronavirus replication due to lethal mutagenesis: Protease inhibitors—interfere with a protein that viruses use to make infectious viral particles; Fusion inhibitors—block the virus from entering the body's cells; Integrase inhibitors—can block an enzyme needed to make copies of itself; Multidrug combinations—combine two or more different types of drugs into one, and/or Pharmacokinetic Enhancers, as well as biological response modifiers, including, for example, interferon (α, β or γ), interleukin-2 and granulocyte-macrophage colony stimulating factor (“GM-CSF”) and the like.

Combination compositions of the present invention may comprise lower doses of an active antiviral while maintaining a level of antiviral activity that is characteristic of a higher dose. As a result, the cytotoxicity typically associated with the administration of an antiviral agent is minimized by the administration of combination compositions of the present invention. Thus, combination compositions may comprise a reduced dosage of an antiviral agent in combination with the anti-CD6 antibody of the present invention to achieve a level of antiviral activity that is greater than that normally required while maintaining an acceptable level of cytotoxicity.

In yet another aspect, the present invention provides for therapeutic methods of treating subjects (e.g., vertebrates, such as humans) by modulating an immune response caused by increase in the expression of interleukins, the method comprising the administration to a subject a therapeutically effective amount of an anti-CD6 antibody comprising SEQ ID NOs 4 and 5 or a sequence having 98% identity thereto including an antibody comprising SEQ ID NOs: 4 and 6 according to the present invention.

In a still further aspect, the present invention provides for a kit for treating a subject against COVID-19 and variants thereof wherein the kit comprises a therapeutically effective amount of an anti-CD6 antibody comprising or consisting of an amino acid sequence of SEQ ID NOs 4 (heavy chain) and 5 (light chain) or an amino acid sequence having 98% identity thereto comprising or consisting of SEQ ID Nos: 4 (heavy chain) and 6 (light chain) according to the present invention.

In a further aspect, the present invention provides for the manufacture of a medicament comprising the anti-CD6 antibody wherein the variable regions of the heavy and light chains comprise an amino acid sequence of SEQ ID NOs 1 and 2 or an amino acid sequence of the variable regions having 98% identity thereto comprising SEQ ID NOs: 1 and 3 according to the present invention.

In another aspect, the present invention provides for the use of the anti-CD6 antibody comprising or consisting of sequence of SEQ ID NO: 4 (heavy chain) and SEQ ID NO: 5 or SEQ ID NO: 6 (light chain) according to the present invention for the treatment of a coronavirus, SARS and MERS-CoV, more specifically for the treatment of an infection of a COVID-19 coronavirus in a subject, mammal or human.

Yet another aspect provides for a treatment method to be used in a subject in need thereof that comprises the intravenous administration of an anti-CD6 monoclonal antibody in a dose range from about 0.5 mg/Kg of body weight to 8 mg/Kg of body weight at least twice in two individual doses up to a maximum of 5 doses spaced 24 to 96 hours apart.

These and other advantages and features of the present invention will be described more fully in a detailed description of the preferred embodiments which follows.

### BRIEF DESCRIPTION OF DRAWINGS

FIG. 1 shows the different stages of the COVID-19 disease (SARS-CoV-2), first an asymptomatic stage with or without detectable virus, Stage 2: non severe asymptomatic period with the presence of virus and finally stage 3: which is the severe symptomatic stage with large viral load and pulmonary edema.

FIG. 2 (a) Remdesirvir; (b) Galidesivir; (c) Camostat; (d) Fingolimod; (e) Favipiravoir; (f) Darunavir/Cobicistat and FIG. 2 Cont. (g) Chloroquine; (h) Baricitinib; (i) Thalidomide; (j) Hydroxychloroquine; (k) Rusolitinib; (l) Umifenovir show the prior art drugs in various clinical trials with their predicted mechanism of action.

FIG. 3 show the mean baseline value of IL-6 comparable in both arms, FIG. 3A shows the A arm and FIG. 3B shows the B arm. Post first infusion a significant decline in mean IL-6 levels was seen in Arm A.

FIG. 4 show the mean baseline value of TNF a comparable in both arms FIG. 4A shows the A arm and FIG. 4B show the B arm. Post first infusion a significant decline in mean IL-6 levels was seen in Arm A.

FIG. 5 refer to concentration of IL-6 in the serum of patients with COVID-19 before treatment with Itolizumab.

FIG. 6 refer to radiological image of a patient with pneumonia due to bacterial infection. FIG. 6A) Before treatment with Itolizumab and FIG. 6 B) After treatment.

FIG. 7 refer to decreasing in C-reactive protein (CRP) concentration media after first dose of Itolizumab. High CRP levels in COVID patients have been associated with more severe disease (see, e.g., Liu et al. J Clin Virol. 2020 June; 127:104370).

FIG. 8 refer to decreasing in ferritin concentration media after first dose of Itolizumab. Ferritin is a key mediator of immune dysregulation; it contributes to cytokine storm, and represents a potential factor influencing the severity of COVID-19 (see, e.g., Vargas-Vargas M and Cortes-Rojo C. Ferritin levels and COVID-19. Rev Panam Salud Publica. 2020; 44:e72).

FIG. 9 shows recovery percentage of patients treated with Itolizumab.

### DETAILED DESCRIPTION OF INVENTION

It is known that COVID-19 and variants thereof can stimulate a cytokine storm in the lungs, such as an increase of IL-2, IL-6, IL-7, GSCF, IP10, MCP1, MIP1A, and TNFα, followed by the edema, dysfunction of the air exchange, acute respiratory distress syndrome, acute cardiac injury and the secondary infection which may lead to death.

The terms Cytokine Storm and Cytokine Release Syndrome are used to refer to an exacerbated immune reaction, in which there is an excessive and uncontrolled release of pro-inflammatory cytokines. Cytokines are a group of low molecular weight proteins that act by mediating complex interactions among lymphoid cells, inflammatory cells and hematopoietic cells. They have highly varied functions that can, however, be classified into a few distinct categories: differentiation and maturation of immune system cells, communication between cells of the immune system and direct effector functions. Cytokines are produced during innate or adaptive immune responses. They bind to specific receptors on the cell membrane where they exert their function, initiating an intracellular signal transduction cascade that alters the gene-expression pattern in a manner that results in target cells producing a specific biological response.

Cytokines are produced by multiple cell types, mainly by immune system cells. One of the most cytokine-producing cells in the innate immune system are macrophages, whereas helper T cells or T CD4+ cells are the ones that mainly produce it in the adaptive or specific immune system. Cytokine production is generally transient, limited to the duration of the stimulus (that is, the foreign agent that induces the immune response).

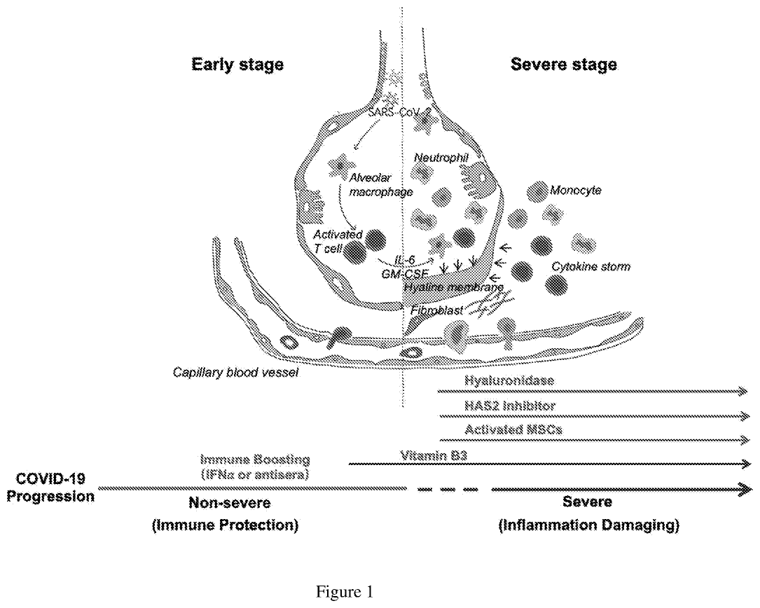

The patient's immune system goes into overdrive to tackle the virus. Th1 cells get activated and release IL6 and GM-CSF cytokine as shown in FIG. 1 which then bring in the macrophages and neutrophils into lung alveoli. These macrophages release more IL6, TNF alpha and IL1 among other pro inflammatory cytokines. These cytokines cause fever by targeting the hypothalamus, and also alveolar edema chocking up the lungs. Patient experiences difficulty in breathing at this stage. The blood vessels around the alveoli become leaky and increased vascular permeability observed. The loss in fluid causes circulatory stress and low blood pressure which then leads to renal failure and finally multi organ failure.

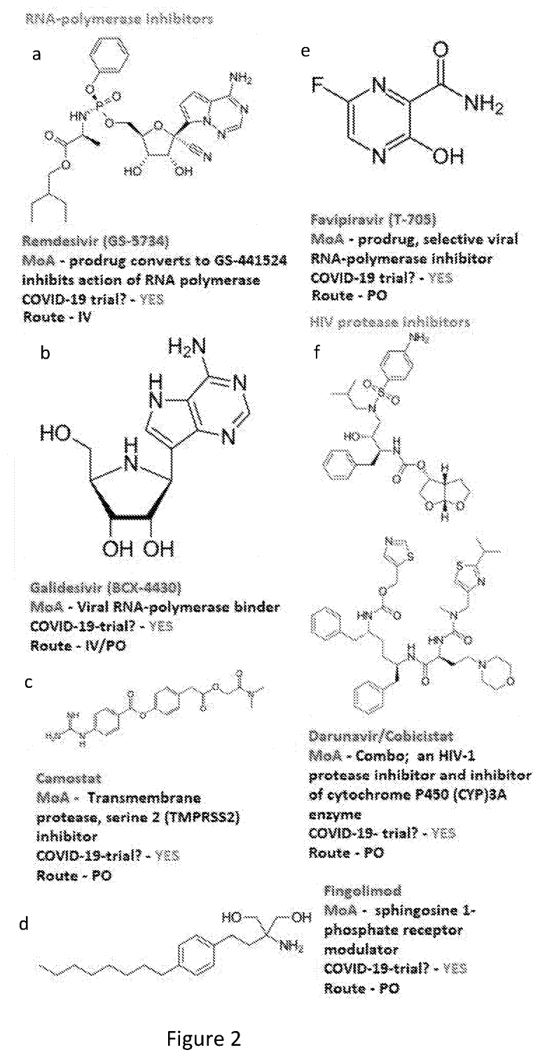

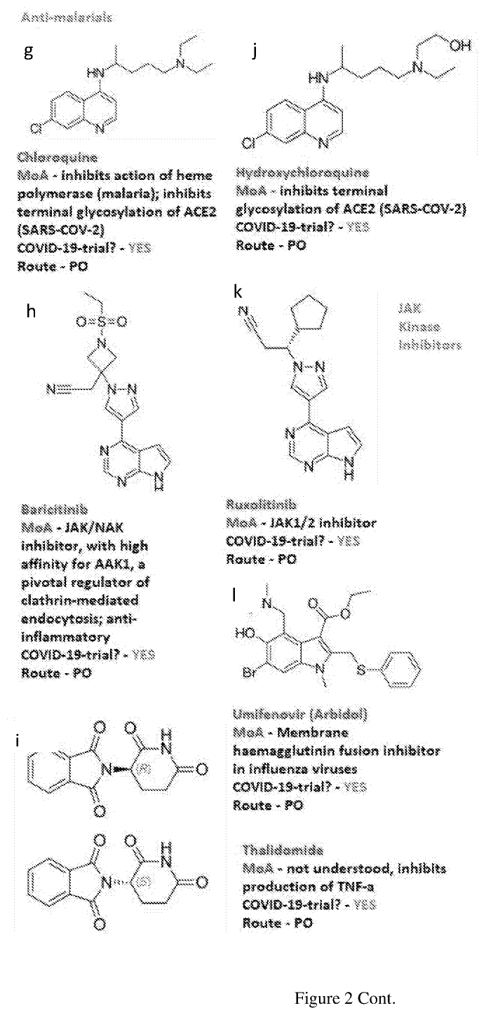

At this time, there are no standard treatment options for COVID infection as this is a very new virus. However, multiple drugs are being tried in combination based on the virus infection pathway mentioned above. FIG. 2 shows the drugs in various clinical trials with their predicted mechanism of action.

The present invention relates to a method of reducing morbidity in a patient infected with SARS-CoV-2 comprising administering to said patient a therapeutic amount of an anti-CD6 antibody. In particular embodiments, the anti-CD6 antibody is a monoclonal antibody. In some embodiments, the anti-CD6 antibody is the humanized monoclonal antibody Itolizumab. In some embodiments, the anti-CD6 antibody is a Fab or fragment. In some embodiments, the patient has a bacterial infection. In some embodiments, the patient infected with SARS-CoV-2 has a comorbidity. In other embodiments, the patient infected with SARS-CoV-2 has more than one comorbidity. In some embodiments, the patient infected with SARS-CoV-2 has more than two comorbidities. In some embodiments, the patient infected with SARS-CoV-2 exhibits no, mild, or less than moderate symptoms. In some embodiments, the patient infected with SARS-CoV-2 exhibits moderate symptoms. In some embodiments, the patient infected with SARS-CoV-2 has severe symptoms. In some embodiments, the patient infected with SARS-CoV-2 exhibits one or more symptoms of cytokine release syndrome (CRS). In some instances of the present invention, Itolizumab is administered to a patient testing positive for COVID-19 and prior to exhibiting one or more morbidities. In some embodiments of the present invention, Itolizumab is administered to a patient in addition to one or more other therapeutic agents.

The antibody of the present invention binds to leukocyte differentiation antigen CD6, which is a glycoprotein that is predominantly expressed in mature peripheral blood lymphocytes and to a lesser extent in mature B cells. It plays a crucial role in cell adhesion, activation, differentiation and survival of lymphocytes (Alonso, R et al. (2008) Hybridoma 27(4):291-301. The CD6 molecule contains three domains in its extracellular part (Sarrias M R et al. (2004) Crit Rev Immunol. 24:1-37) and the binding site to its ligand, the ALCAM (Activated Leukocyte-Cell Adhesion Molecule) molecule, is located on domain 3 (Bodian D L et al. (1997) Biochemistry 36: 2637-2641).

The humanized mAb itolizumab of the present invention recognizes and binds to the Scavenger receptor cysteine-rich (SRCR) domain 1 (D1) of CD6 and does not interfere with the binding of the CD6 to its ligand ALCAM, therefore, it does not produce the immunosuppressive effects reported with other monoclonal antibodies used against the same target.

The use of anti-CD6 antibodies, including non-depleting anti-CD6 antibodies as described in the present invention rapidly alleviates the symptoms and signs related to the cytokine storm or the cytokine release syndrome in treated patients. The reduction of these symptoms results in a substantial clinical improvement of critically ill patients and allows for a better management of the patient by the intensive care unit (ICU) team. A particular advantage of the proposed treatment is that it does not induce immunodeficiency in the patient, unlike other conventional therapies based on steroids or other immunosuppressants. The preservation of a certain degree of immunocompetence in treated patients reduces the possibility of emergencies due to other opportunistic infections, very common in intensive care.

The term “non-depleting” anti-CD6 antibody, as used herein means a monoclonal antibody specifically directed to CD6, that upon binding, does not induce antibody-dependent cellular cytotoxicity (ADCC), complement-dependent cellular cytotoxicity (CDC) or does not otherwise promote lysis and death of a cell expressing CD6.

To date, there are no previous reports on the use of mAbs against the CD6, particularly the capacity of these antibodies to inhibit the activation of the immune system and the uncontrolled production of cytokines, without causing immunodeficiency. The use of Itolizumab is of great value in combating the toxicity derived from the cytokine storm, allowing the body to continue fighting the viral infection due to a coronavirus.

Unless otherwise defined, all other scientific and technical terms used in the description, figures and claims have their ordinary meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purposes of describing particular embodiments only and is not intended to be limiting. As used in this specification and the appended claims, the singular forms “a”, “an” and “the” include plural referents unless the content clearly dictates otherwise. Thus, for example, reference to “a cell” includes a combination of two or more cells, and the like.

The word “about” as used herein refers to a value being within an acceptable error range for the particular value as determined by one of ordinary skill in the art, which will depend in part on how the value is measured or determined, i.e., the limitations of the measurement system. For example, “about” can mean within one, or more than one standard deviation, per the practice in the art. The term “about” is also used to indicate that the amount or value in question may be the value designated or some other value that is approximately the same. The phrase is intended to convey that similar values promote equivalent results or effects as disclosed herein. In this context “about” may refer to a range above and/or below of up to 10%. The word “about” refers in some embodiments to a range above and below a certain value that is up to 5%, such as up to up to 2%, up to 1%, or up to 0.5% above or below that value.

The term “activity”, “biological activity”, therapeutical activity” or “functional activity”, as used herein refers to an activity exerted by Itolizumab, as determined in vivo, or in vitro, according to standard techniques.

The term “anti-virally-effective amount” as used here means an amount of Itolizumab compound according to the invention that results in treatment, prophylaxis, slowing of spread of the coronavirus including COVID-19 or of manifestations of coronavirus, prevention of infection of others and/or improvement in patient condition.

The term “cytokine” as used herein is meant to include any one of the group of hormone-like mediators produced by T and B-lymphocytes. Representative cytokines include but are not limited to Interleukin-1 (IL-1), IL2, IL3, IL4, IL-5, IL-6, IL-7, IL-8, IL-9, IL-10, IL-11, IL-12, IL-13, IL-15, IL-18, Interferon gamma (IFN-.gamma.), Tumor Necrosis Factor alpha (TNF-.alpha.), and Transforming Growth Factor-beta (TGF-.beta.).

The term “lymphocyte” as used herein has the normal meaning in the art, and refers to any of the mononuclear, non-phagocytic leukocytes, found in the blood, lymph, and lymphoid tissues, e.g., B and T lymphocytes.

Variants of the COVID-19 virus may include changes in the amino acids found in the spike protein of the virus, but also in the ORF protein region. For example, the B.1.1.7 variant has a notable mutation N501Y. Other mutations have been found to be more infective including A222V, E484K, S477N and K417N/T. As the virus spreads and more people are infected additional variants are inevitable

The terms “patient” and “subject” as used herein are used interchangeably and refer to human patients.

The terms “treating” or “treatment” as used herein includes the administration of the antibody compositions, compounds or agents of the present invention to prevent or delay the onset of the symptoms, complications, or biochemical indicia of a disease, alleviating the symptoms or arresting or inhibiting further development of the disease, condition, or disorder (e.g., cancer, metastatic cancer, or metastatic breast cancer). Treatment can be prophylactic (to prevent or delay the onset of the disease, or to prevent the manifestation of clinical or subclinical symptoms thereof) or therapeutic suppression or alleviation of symptoms after the manifestation of the disease.

The term “well tolerated” as used herein refers to the absence of adverse changes in health status that occur because of the treatment and would affect treatment decisions.

The phrase “modulating an immune response” or “modulation of an immune response” as used herein includes downregulation, inhibition or decreasing an immune response as defined herein.

The term “anti-CD6 antibody” as used herein is an antibody that bind specifically to SRCR domain 1 (D1) of human CD6 (hCD6) and that does not interfere with the activated leukocyte cell adhesion molecule (ALCAM) binding to CD6.

The CD6 mAbs (monoclonal antibodies) used in the present invention can be administered as part of pharmaceutical compositions containing the mAb as active agent and as suitable excipient a physiological buffer similar to the one used to formulate the mAbs to be administered by intravenous route. Particularly, the sequences of the Itolizumab are described in U.S. Pat. Nos. 6,572,857 and 8,524,233, the contents of which are incorporated by reference here in below table 1.

Protein sequence of Itolizumab

| SEQ | | |
|---|---|---|
| ID | | |
| NO. | TDESCRIPION | SEQUENCE |
| 1 | Variable | EVQLVESGGGLVKPGGSLKLSCAAS |
| | region of | GFKFSRYAMSWVRQAPGKRLEWVAT |
| | Heavy | ISSGGSYIYYPDSVKGRFTISRDNV |
| | Chain | KNTLYLQMSSLRSEDTAMYYCARRD |
| | | YDLDYFDSWGQGTLVTVSS |
| 2 | Variable | DIQMTQSPSSLSASVGDRVTITCKA |
| | region of | SRDIRSYLTWYQQKPGKAPKTLIYY |
| | Light | ATSLADGVPSRFSGSGSGQDYSLTI |
| | Chain | SSLESDDTATYYCLQHGESPFTLGS |
| | | GTKLEIK |
| 3 | Variable | DIQMTQSPSSLSASVGDRVTITCKA |
| | region of | SRDIRSYLTWYQQKPGKAPKTLIYY |
| | Light | ATSLADGVPSRFSGSGSGQDYSLTI |
| | Chain | SSLESDDTATYYCLQHGESPFTFGS |
| | | GTKLEIKRA |
| 4 | Heavy | EVQLVESGGGLVKPGGSLKLSCAAS |
| | Chain | GFKFSRYAMSWVRQAPGKRLEWVAT |
| | | ISSGGSYIYYPDSVKGRFTISRDNV |
| | | KNTLYLQMSSLRSEDTAMYYCARRD |
| | | YDLDYFDSWGQGTLVTVSSASTKGP |
| | | SVFPLAPSSKSTSGGTAALGCLVKD |
| | | YFPEPVTVSWNSGALTSGVHTFPAV |
| | | LQSSGLYSLSSVVTVPSSSLGTQTY |
| | | ICNVNHKPSNTKVDKKVEPKSCDKT |
| | | HTCPPCPAPELLGGPSVFLFPPKPK |
| | | DTLMISRTPEVTCVVVDVSHEDPEV |
| | | KFNWYVDGVEVHNAKTKPREEQYNS |
| | | TYRVVSVLTVLHQDWLNGKEYKCKV |
| | | SNKALPAPIEKTISKAKGQPREPQV |
| | | YTLPPSRDELTKNQVSLTCLVKGFY |
| | | PSDIAVEWESNGQPENNYKTTPPVL |
| | | DSDGSFFLYSKLTVDKSRWQQGNVF |
| | | SCSVMHEALHNHYTQKSLSLSPGK |
| 5 | Light | DIQMTQSPSSLSASVGDRVTITCKA |
| | Chain | SRDIRSYLTWYQQKPGKAPKTLIYY |
| | | ATSLADGVPSRFSGSGSGQDYSLTI |
| | | SSLESDDTATYYCLQHGESPFTLGS |
| | | GTKLEIKRTVAAPSVFIFPPSDEQL |
| | | KSGTASVVCLLNNFYPREAKVQWKV |
| | | DNALQSGNSQESVTEQDSKDSTYSL |
| | | SSTLTLSKADYEKHKVYACEVTHQG |
| | | LSSPVTKS |
| 6 | Light | DIQMTQSPSSLSASVGDRVTITCKA |
| | Chain | SRDIRSYLTWYQQKPGKAPKTLIYY |
| | | ATSLADGVPSRFSGSGSGQDYSLTI |
| | | SSLESDDTATYYCLQHGESPFTFGS |
| | | GTKLEIKRARTVAAPSVFIFPPSDE |
| | | QLKSGTASVVCLLNNFYPREAKVQW |
| | | KVDNALQSGNSQESVTEQDSKDSTY |
| | | SLSSTLTLSKADYEKHKVYACEVTH |
| | | QGLSSPVTKSFNRGEC |

Notably, CD6 is constitutively expressed mainly on effector T cells (Teff cells) and rarely expressed on regulatory T cells (Tregs). CD6 stimulates the immune response and is overexpressed after lymphocyte activation. CD6 homes inflammatory cells to lesion, and its ligand ALCAM is upregulated after activation and in inflamed tissues.

CD6 and viruses: CD6 is overexpressed on T-cells during chronic SIV infection, with impaired anti-viral responses, and is associated with SW disease progression. HTLV-1 induces overexpression of ALCAM facilitating the trafficking of infected lymphocytes through the blood-brain barrier. ALCAM is increased on HIV+ monocytes and anti-ALCAM antibodies and the CCR2/CCR5 dual inhibitor reduce their transmigration.

Itolizumab is an anti-CD6 humanized IgG1 mAb. Itolizumab is an immune modulatory molecule without target cell depletion (no cytopenias). Itolizumab accumulates in the inflamed lesion. Itolizumab has a potent anti-inflammatory effect reducing the production of pro-inflammatory cytokines IL-6, TNF, IFNγ, IL-17 and IL-1.

It is shown herein that Itolizumab in COVID-19 patients has the ability to control the pro-inflammatory cytokine storm syndrome, by immunomodulation of T_{eff} function (Th1/Th2/Th17 cells) and prevent trafficking of lymphocytes to the inflammation site (disrupting ALCAM-CD6 interaction), sparing Tregs and preserving the anti-viral response. This is expected to reduce the morbidity and mortality of patients with COVID-19 positivity associated with cytokine release syndrome.

Notably, higher percentage of CD14+CD16+ inflammatory monocyte exists in peripheral blood of COVID-19 patients. The percentage of CD14+CD16+ monocyte is much higher in patients exhibiting ARDS. Moreover, a significantly higher expression of IL-6 is secreted from these inflammatory monocytes especially in patients with ARDS. This increase in IL-6+ monocytes is theorized to be related to the cytokine storm caused by monocytes that can migrate to the lung area. Thus, in COVID-19 patients these activated immune cells may enter the pulmonary circulation in large quantities and exhibit an immune damaging role in ARDS. Thus, the present invention provides for a novel approach to reduce inflammatory cytokine proteins, which can include interleukins IL-1, IL-6 and IL-2, and interferon-gamma and to treat ill individuals with complications of hyper-inflammation due to COVID-19 and variants thereof.

In certain embodiments, methods of the present invention include preventing acute respiratory distress syndrome (ARDS) in a COVID-19 patient wherein the method consists of treating the patient with a therapeutic amount of an anti-CD6 antibody. In some embodiments, methods of the present invention decrease ARDS mortality rate in patients with COVID-19. In certain embodiments, the present invention provides methods of decreasing the hospital stay of a patient infected with SARS-CoV-2, said method comprising administering to the patient a therapeutic amount of an anti-CD6 antibody. In some embodiments, methods of the present invention prevent myocarditis in a patient with COVID-19. In some embodiments, methods of the present invention are used to lower the ICU mortality odds ratio of COVID-19 patients.

In certain embodiments, methods of the present invention include preventing infectious diseases caused by bacterial agents in patients wherein the method comprises of treating the patient with a therapeutic amount of an anti-CD6 antibody. These bacterial agents include but are not limited to group A streptococci and pneumococci.

In some embodiments of the present invention, Itolizumab is administered to a patient in addition to one or more other therapeutic agents. In certain embodiments, the additional therapeutic agent is chosen from the group consisting of vaccines, antivirals, antibodies, immunotherapies, immunomodulators, cytokine inhibitors, anti-coagulants, complement inhibitors, microbiome regulators, and antimalarial. In some embodiments of the present invention, the additional agent is a vaccine. In some embodiments the vaccine is chosen from the list consisting of BNT-162b2, ChAdOx1 nCoV-19, or mRNA-1273. In some embodiments of the present invention, the additional agent is an antiviral. In certain embodiments, the antiviral is selected from the list consisting of a viral neuraminidase inhibitor (for example Oseltamivir or Zanamivir), a viral polymerase inhibitor (for example Ribavirin) or M2 ion-channel blocker (for example amantadine or rimantadine), remdesivir, biomedivir, favipiravir, or nanomedivir. In some embodiments of the present invention, additional therapeutic agent is Phase II inhibitors such as T-705 Toyama Chemical Co. (oral polymerase inhibitor), Peramivir™, BioCryst (IV/IM neuraminidase inhibitor), or phase I inhibitors such as CS-8958, Biota/Daiichi Sankyo (inhaled neuraminidase inhibitor). In some embodiments of the present invention, the additional agent is an antibody. In certain embodiments, the additional agent is a neutralizing antibody. In some embodiments, the neutralizing antibody is LY-CoV555 or REGN-COV2. In some embodiments of the present invention, the additional agent is an immunotherapeutic agent. In certain embodiments of the present invention, convalescent plasma is the additional agent. In some embodiments of the present invention, the additional agent is an immunomodulator. In certain embodiments, the immunomodulator is a glucocorticoid. In some embodiments, the immunomodulator is a kinase inhibitor. In some embodiments, the additional agent is ciclesonide. In certain embodiments, the additional agent is baricitinib. In some embodiments of the present invention, the additional agent is ruxolitinib. In some embodiments of the present invention, the additional agent is a cytokine inhibitor. In some embodiments, the cytokine inhibitor is chosen from the list consisting of tocilizumab, sarilumab, canakinumab, lenzilumab. In some embodiments of the present invention, the additional agent is a stem cell therapy. In some embodiments, the stem cell therapy is remestemcel-L or emiplacel. In some embodiments of the present invention, the additional agent is an anti-coagulant. In certain embodiments, the additional agent is an anti-thrombotic. In some embodiments, the methods of the present invention are used with an additional agent selected from the list comprising rivaroxaban, sulodexide, apixaban, heparin, and aspirin. In some embodiments of the present invention, the additional agent is a complement inhibitor. In some embodiments, the complement inhibitor is selected from eculizumab or ravulizumab. In some embodiments of the present invention, the additional agent is a microbiome regulator. In certain embodiments, the additional agent is EDP-1815. In some embodiments of the present invention, the additional agent is an antimalarial. In certain embodiments, the antimalarial is selected from chloroquine and hydroxychloroquine. In some embodiments, this invention also includes antiviral compositions comprising an anti-CD6 antibody according to the present invention administered with one or more other antiviral agents, such as Reverse transcriptase (RT) inhibitors, Nucleoside analogs, Fusion inhibitors and/or Integrase inhibitors. In certain embodiments of the present invention, the reverse transcriptase inhibitors include, but are not limited to clevudine, telbivudine, tenofovir, dipovoxil, ganciclovir, lobucavir, famciclovir, and penciclovir. In some embodiments, the nucleoside analogs include, but are not limited to, abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC, pharmaceutically acceptable salts thereof, and combinations thereof. In certain embodiments of the present invention, the fusion inhibitors include, but are not limited to, enfuvirtide (“Fuzeon”), T-20, PRO 140, vicriviroc, and maraviroc. In other embodiments of the present invention, the integrase inhibitors include, but are not limited to, globoidnan A, L-000870812, S/GSK1349572, S/GSK1265744, Raltegravir and Elvitegravir.

Therapeutic options with Itolizumab for patients infected with COVID-19 including subjects with symptomatic or asymptomatic COVID-19 infections are mentioned in Table 2.

TABLE 2
All options may also include Azithromycin and anti-
retroviral replication drugs (Anti-RNA polymerase)

| Sr. | Compound or | |
|---|---|---|
| No. | Combination | Rationale |
| 1. | Itolizumab alone | Itolizumab has a potent anti-inflammatory effect reducing the |
| | | production of pro-inflammatory cytokines IL-6, TNF, IFNγ, IL- |
| | | 17, IL-2 and IL-1. A significantly higher expression of IL-6 is |
| | | secreted from inflammatory monocytes especially in patients |
| | | with ARDS. This increase in IL-6 + monocytes can be related to |
| | | the cytokine storm caused by monocytes that can migrate to the |
| | | lung area. Thus, in COVID-19 patients these activated immune |
| | | cells may enter the pulmonary circulation in large quantities and |
| | | exhibit an immune damaging role in ARDS. |
| 2. | Itolizumab + Cytosorb | Cytosorb will mop off multiple pro inflammatory cytokines |
| | | systemically. Has been used extensively in the treatment of sepsis |
| | | and tried in a few COVID positive cases with cytokine release |
| | | syndrome. |
| 3. | Itolizumab + | Hydroxychloroquine mediated inhibition of autophagy prevents |
| | Hydroxychloroquine | immune activation of different cell types, which inhibits cytokine |
| | | production. It is also associated with inhibition of the terminal |
| | | glycosylation of ACE2. Has been tried in combination with other |
| | | drugs in COVID positive cases with cytokine release syndrome |
| 4. | Itolizumab + IL6 | Considering very high level of IL6 in complicated COVID 19 |
| | receptor antagonist | cases, IL6 receptor antagonist would immediately block IL6 |
| | | mediated signalling and Itolizumab will prevent further |
| | | activation and proliferation of effector T cells and hence supress |
| | | further immune activation. Anti IL6 has been used in |
| | | combination with other drugs in COVID positive cases with |
| | | cytokine release syndrome |
| 5. | Itolizumab + JAK | JAK inhibitors will block signalling through cytokine receptors |
| | inhibitor | as an immediate step and is therefore anti inflammatory. In |
| | | addition, JAK inhibitors with high affinity of AAK1 is a pivotal |
| | | regulator of clathrin mediated endocytosis. |

Within the scope of this invention is the administration of Itolizumab prophylactically. Administration of Itolizumab of the present invention can occur prior to the manifestation of symptoms of the COVID-19 virus, such that the virus is prevented or, alternatively, delayed in its progression. The prophylactic methods of the present invention can be carried out in a similar manner to the therapeutic methods described herein, although dosage and treatment regimens may differ. Thus, it is considered beneficial to administer Itolizumab prophylactically as a preventive method to subjects showing no evidence of the virus.

This present invention includes treatment or therapy of patients infected with COVID-19 including subjects with symptomatic or asymptomatic COVID-19 infections.

The Itolizumab mAb of the present invention is administered in a manner compatible with the dosage formulation and in such amount as will be therapeutically effective. The quantity to be administered depends on the subject to be treated, capacity of the subject's immune system to generate a cellular immune response, and degree of protection desired. Precise amounts of active ingredient administration depend on the judgment of the practitioner and are peculiar to each individual. However, Itolizumab can be delivered at effective amount of about 0.1 to 25 mg/Kg of body weight/week, more preferably about 0.5 mg/kg to about 10 mg/kg per body weight, and most preferably from about 1 to 3 mg/kg per body weight per week delivered by intravenous (IV) administration.

In one embodiment of the present invention, patients are screened for co-morbidities and treated with a single dose of Itolizumab. In one embodiment COVID-19 patients with co-morbidities are treated with 200 mg Itolizumab by dosage of 3.2 mg/kg. In one embodiment, the treatment is repeated between one and fourteen days later.

Combination compositions of the present invention may comprise lower doses of the active antiviral while maintaining a level of antiviral activity that is characteristic of a higher dose. As a result, the cytotoxicity typically associated with the administration of an antiviral agent is minimized by the administration of combination compositions of the present invention. Combination compositions may comprise a reduced dosage of an antiviral agent in combination with the Itolizumab of the present invention to achieve a level of antiviral activity that is greater than that normally required while maintaining an acceptable level of cytotoxicity.

Some methods of the present invention include first testing a patient for COVID-19 and for patients positive for COVID-19 administering a therapeutic dose of an anti-CD6 antibody. Some methods of the present invention provide selection criteria for determining which COVID-19 patients to treat with a therapeutic dose of Itolizumab. Some methods of the present invention include administering a COVID-19 test and assessing the patient for worsening lung involvement and administering an anti-CD6 antibody to patients who test positive for COVID-19 and have worsening lung involvement. In some methods of the present invention determining if lung involvement is worsening includes assessing the patient to find at least one of the following: a) worsening oxygen saturation; b) decrease in PaO₂; c) need to increase FiO₂; d) unstable SO₂; e) increased need for ventilator support; f) new need for ventilator support; f) increase in the number and/or extent of consolidation lung areas. In some methods, the oxygen saturation worsens by more than 1 percentage point. In some methods of the present invention, the oxygen saturation of a patient worsens by >3 percentage points. In some embodiments the PaO₂ decrease is >10%.

In some embodiments of the present invention, methods include selecting patients with COVID-19 and suspected macrophage activation syndrome and administering a therapeutic dose of Itolizumab. In some embodiments, a patient who tests positive for COVID-19 and needs oxygen therapy is administered a therapeutic dose of Itolizumab. In some embodiments, the present invention consists of administering to a COVID-19 patient with confirmed multifocal interstitial pneumonia a therapeutic dose of Itolizumab. In some embodiments, a patient positive for COVID-19 who also needs oxygen therapy to maintain saturation of O₂>93% is administered a therapeutic dose of an anti-CD6 antibody. In some embodiments, methods of the present invention are used to treat a patient who tests positive for COVID-19 and has one or more of: a) wheezing or irregular speech; b) respiratory frequency greater than 22 breaths per minute; c) P02: Partial arterial oxygen pressure <65 mm Hg; d) worsening radiological image; e) fever >=38° C.; f) reduction of initial values of hemoglobin, platelets or neutrophils; g) Hb<90 g/L; h) platelets <100×109/L; i) neutrophils <1×109/L; j) leukocytes <4×109/L; k) decreased erythrocyte sedimentation in mismatch with PCR (low erythrocyte sedimentation and PCR increases or does not change); l) increased initial value of triglycerides or triglycerides greater than 3 mmol/L; m) increased initial ferritin value from 500 ng/ml or absolute ferritin value >=2000 ng/ml; n) transaminase aspartate-aminotransferase >=30 IU/L; o) increase in dimer D; p) fibrinogen <2.5 g/L; q) onset of neurological manifestations. In some embodiments, methods of the present invention include using exclusion criteria to select patients for treatment with an anti-CD6 antibody. In some embodiments the exclusion criteria include one or more of: pregnancy, nursing, and one or more adverse events.

Some embodiments of the present invention include determining how long to treat a patient, and at what dose, by measuring one or more primary outcome measurements selected from the list consisting of: reduction or deterioration of lung function, patient rate without the need to increase FiO₂ to keep SO₂ stable and without the need for intubation; rate of patients decreasing positive pressure values at the end of exhalation (PPFE)). In some embodiments the measurement time of these outcomes is between one and 7 days. Some embodiments of the present invention include determining how long to treat a patient, and at what dose, by measuring one or more secondary outcome measurements selected from the list consisting of: 1. need for endo-tracheal intubation; 2) increase in non-invasive or invasive mechanical ventilation rate when respiratory failure occurs; 3) non-invasive mechanical ventilation failure; 4) duration of mechanical ventilation or time to end of mechanical ventilation; 5) rate of patient mortality; 6) patient serum concentration of IL1, IL6 and TNF alpha; 7) HSH score parameters (Temperature, visceromegaly, cytopenia, triglycerides, fibrinogen, ferritin, AAT (GOT)); 8) C-reactive protein, and absolute lymphocyte count (positive or negative).

Some methods of the present invention include administering to a moderately ill COVID-19 patient a therapeutic dose of an anti-CD6 antibody. Some methods of the present invention include administering to a COVID-19 patient of advanced age a therapeutic dose of an anti-CD6 antibody. In some embodiments the patient is 64 years or older. In some embodiments, the anti-CD6 antibody is Itolizumab. In some embodiments of the present invention, hospitalized patients positive for COVID-19 are treated with Itolizumab combined with anti-viral therapies and compared to a control group that received standard of therapy anti-viral care and no Itolizumab. In some embodiments of the present invention, patients are assessed for COVID-19 by real-time transcriptase polymerase chain reaction (RT-PCR).

Statistical significance of treatments is determined through mean and standard deviations or median and interquartile ranges (depending on the distribution of each variable). Wilcoxon rank sum test is applied to continuous variables, and chi-square or Fisher's exact test are used for categorical variables. In some embodiments, the present invention provides selecting a control group from the same hospitalized patient group of COVID-19 patients that did not receive immunomodulatory therapy. Controls are treated with lopinavir/ritonavir, chloroquine, interferon α2B and LMWH. Control subjects are well matched regarding age, comorbidities and severity of the disease. Odds ratio for disease progression and mortality are estimated for Itolizumab vs control. Comorbidities include hypertension, dementia, malnutrition, cardiac disease, diabetes mellitus, chronic obstructive pulmonary disease.

Each 5 mL vial contains Itolizumab 25 mg (r-DNA origin) which has solution for iv infusion, colourless and transparent solution. Itolizumab is packed in 6R clear glass vial (USP type 1) closed with a chlorobutyl rubber stopper and sealed with flip-off seals. Itolizumab Injection is a preservative-free solution, supplied in single-use vial for IV infusion. Prior to use, the solution in the vial inspected visually for particulate matter and discolouration. If visible opaque particles, discolouration, or other foreign particulates are observed, the vial was to be discarded and the solution not to be administered to the patient. Appropriate volume of Itolizumab injection was added to 250 mL normal saline and mixed gently. This diluted infusion bag could be stored at room temperature or refrigerated at 2° C. to 8° C. protected from light, and it is stable up to 10 hours at room temperature. The infusion solution was allowed to reach at room temperature prior to administration to patients. The Itolizumab infusion was administered over a period of not less than 120 minutes and using an infusion set with an in-line, sterile, non-pyrogenic, low protein-binding filter (pore size of 1.2 μm or less). Approximately 50 mL of infusion solution was administered during the first hour, followed by remaining solution (approximately 200 mL) in the next hour. The infusion period could be extended up to 8 hours for medical reasons. Itolizumab could not be infused concomitantly in the same IV line with any other agents. Any unused portion of the infusion solution was not to be stored for reuse.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. The efficient dosages and the dosage regimens for the anti-CD6 monoclonal antibodies used in the present invention depend on the severity of the COVID-19 disease and variants thereof and may be determined by the medical practitioners. In one embodiment, the anti-CD6 monoclonal antibody is administered by infusion in a weekly dosage. Such administration may be repeated, e.g., 1 to 8 times, such as 2 to 4 times, or 3 to 5 times. In the alternative, the administration may be performed by continuous infusion over a period of from 2 to 24 hours, such as, from 2 to 12 hours.

An exemplary, non-limiting range for a therapeutically effective amount of the anti-CD6 monoclonal antibody used in the present invention is about 0.01-100 mg/kg per subject body weight, such as about 0.01-50 mg/kg, for example about 0.01-25 mg/kg. In some embodiments, the ideal weight for patient's height is used to determine dose. In some embodiments, more than one dose is given to a subject. In some embodiments, a larger initial dose is given to the patient. In some embodiments, a second dose is administered after one week. In some embodiments, a second dose is administered after two weeks. In some embodiments, the second dose is the same strength as the first dose. In some embodiments, the second dose is three-fourths or less of the initial dose. In some embodiments, the second dose is half of the initial dose. In some embodiments, a third treatment is administered. In some embodiments, therapeutically effective amounts of an anti-CD6 monoclonal antibody are administered every two weeks until a patient is determined to be recovering or discharged from the hospital. In some embodiments, the doses are either 0.8 mg/kg or 1.6 mg/kg. In some embodiments, the doses administered are 1.6 mg/kg and 0.8 mg/kg. Exemplary, non-limiting doses for a therapeutically effective amount of the anti-CD6 monoclonal antibody used in the present invention are 0.8 mg/kg and 1.6 mg/kg. A medical professional having ordinary skill in the art may readily determine and prescribe the effective amount of the pharmaceutical composition required. For example, a physician could start doses of the anti-CD6 monoclonal antibody at levels lower than that required in order to achieve the desired therapeutic effect and gradually increase the dosage until the desired effect is achieved.

In one embodiment, the anti-CD6 monoclonal antibody is administered by infusion in a weekly dosage of from 0.1 to 50 mg/kg per subject body weight, such as, from 0.5 to 3 mg/kg. Such administration may be repeated, e.g., 1 to 8 times, such as 2 to 4 times, or 3 to 5 times. In the alternative, the administration may be performed by continuous infusion over a period of from 2 to 24 hours, such as, from 2 to 12 hours. In one embodiment, the anti-CD6 monoclonal antibody is administered in a weekly dosage. In some of these embodiments the dosage of from 50 mg to 350 mg of Itolizumab is administered up to 7 times, such as from 2 to 4 times. In some embodiments, the anti-CD6 antibody is administered biweekly. The administration may be performed by continuous infusion over a period of from 2 to 24 hours, such as, from 2 to 12 hours. Such regimen may be repeated one or more times as necessary, for example, after one week or after two weeks.

Most assessments and study procedures were as per standard treatment protocols established by participating study centers and COVID-19 disease management guidelines from ICMR. Treatment Emergent Adverse Events (TEAEs) were graded according to common terminology criteria for Adverse Events (CTCAE) criteria's (v5.0) during treatment and up to 30 days after the first treatment dose.

The Examples which follow are set forth to aid in understanding the invention but are not intended to and should not be construed to limit its scope in any way. The Examples do not include detailed descriptions for conventional methods employed in the assay procedures. Such methods are well known to those of ordinary skill in the art and are described in numerous publications including by way of examples.

### Example 1

Patients who tested positive for virological diagnosis of SARS-CoV2 infection (RT-PCR) were randomized for enrolment from various centers in India.

Major Inclusion Criteria for patients were:
Confirmed virological diagnosis of SARS-CoV2 infection (RT-PCR)
Hospitalized due to clinical worsening of COVID-19 infection
Oxygen saturation at rest in ambient air ≤94%
Patients who are in moderate to severe ARDS as defined by PaO₂/Fio₂ (Partial pressure of Oxygen/Fraction of Inspired Oxygen) ratio of <200 or more than 25% deterioration from the immediate previous value.
Baseline serum ferritin level ≥400 ng/mL or IL-6 levels greater than 4 times ULN, if known

Major Exclusion criteria for patients were:
Known severe allergic reactions to monoclonal antibodies
Active tuberculosis (TB) infection; History of inadequately treated tuberculosis or latent tuberculosis
Patient on invasive mechanical ventilator support.
Have received oral anti-rejection or immune-suppressive drugs within the past 6 months
Participating in other drug clinical trials like using anti-IL-6 therapy like tocilizumab (participation in COVID-19 anti-viral trials may be permitted if approved by Sponsor)
Patient on treatment of anti-IL-6 or plasma therapy as a part of supportive care
Pregnant or breastfeeding, or positive pregnancy test in a pre-dose examination
Patients with known history of Hepatitis B, Hepatitis C or HW
Absolute Neutrophils count (ANC)<1000/mm³; Platelet count <50,000/mm³; Absolute Lymphocyte count (ALC): <500/mm³

The 6 week long, study of treatment was conducted in two arms in approximately 36 patients.

Treatment Arm A: Best supportive care+Itolizumab (SEQ ID Nos: 4 and 5); wherein; the best supportive care was given as per the institution's protocol was administered (such as antivirals/antibiotics/hydroxychloroquine; oxygen therapy, etc) along with Itolizumab. Premedication with Hydrocortisone 100 mg i.v (or equivalent short acting glucocorticoid) and Pheniramine 30 mg per i.v. about 30±10 minutes was administered in Arm A patients prior to infusion.

Treatment Arm B: Best supportive care; wherein; the best supportive care to be given as per the institution's protocol was administered (such as antivirals/antibiotics/hydroxychloroquine; oxygen therapy, etc) without Itolizumab.

All eligible patients entering into the study were randomized in 2:1 ratio to receive the treatment A (best supportive care+Itolizumab) or treatment B (best supportive care) respectively. A computer derived randomization schedule was generated using appropriate system e.g. SAS to assign patient to treatment groups. Randomization was central and remote telephone and computer-based email systems was used to distribute randomization schedule to the site. If the patient was randomized to Arm A, and was not initiated Itolizumab or not administer one full infusion, patient was then not considered randomized. The same randomization code was used for the subsequent patient in that particular site.

Supportive care including oxygen therapy, heparin, antivirals, antibiotics, short-term steroids and vitamins were allowed. Prior and concomitant treatment with the oral anti-rejection or immune-suppressive drugs (continuously/regularly in the last 6 months), IL-6 therapy like tocilizumab, convalescent plasma was not permitted.

Itolizumab Injection was in a preservative-free solution, supplied in single-use vial for IV infusion. Prior to use, the solution in the vial was inspected visually for particulate matter and discolouration. If visible opaque particles, discolouration, or other foreign particulates are observed, the vial was discarded and the solution was not administered to the patient. Appropriate volume of Itolizumab injection was added to 250 mL normal saline and mixed gently. This diluted infusion bag could be stored at room temperature or refrigerated at 2° C. to 8° C. protected from light, and it is stable up to 10 hours at room temperature. The infusion solution was allowed to reach at room temperature prior to administration to patients.

The Itolizumab infusion was administered over a period of not less than 120 minutes and using an infusion set with an in-line, sterile, non-pyrogenic, low protein-binding filter (pore size of 1.2 μm or less). Approximately 50 mL of infusion solution was administered during the first hour, followed by remaining solution (approximately 200 mL) in the next hour. The infusion period could be extended up to 8 hours for medical reasons. Itolizumab could not be infused concomitantly in the same IV line with any other agents. Any unused portion of the infusion solution was not stored for reuse.

Most assessments and study procedures were as per standard treatment protocols established by participating study centers and COVID-19 disease management guidelines from ICMR. Treatment Emergent Adverse Events (TEAEs) were graded according to common terminology criteria for Adverse Events (CTCAE) criteria's (v5.0) during treatment and up to 30 days after the first treatment dose.

As stated above, a total of 36 patients were screened, of which 4 patients were considered screen failures. Of the 32 patients who met inclusion criteria or did not met exclusion criteria, 2 patients intended for Itolizumab treatment discontinued prior to completion of the first dosing and were replaced as per protocol. The median age of patients in Arm A was 50.5 years and 49.5 years in Arm B. The majority of them were male in both treatment arms and all patients were Asian. All patients in Arm A and majority of patients in Arm B (all patients who were alive) completed the study; this included 16 (80%) and 6 (60%) patients in Arms A and B, respectively who were discharged early. The most common reason for study discontinuation in Arm B was death (n=3). There were no deaths in Arm A. Arm A patients received best supportive care along with 1.6 mg/kg per two-week dosage of Itolizumab; wherein Arm B patients received Best supportive care.

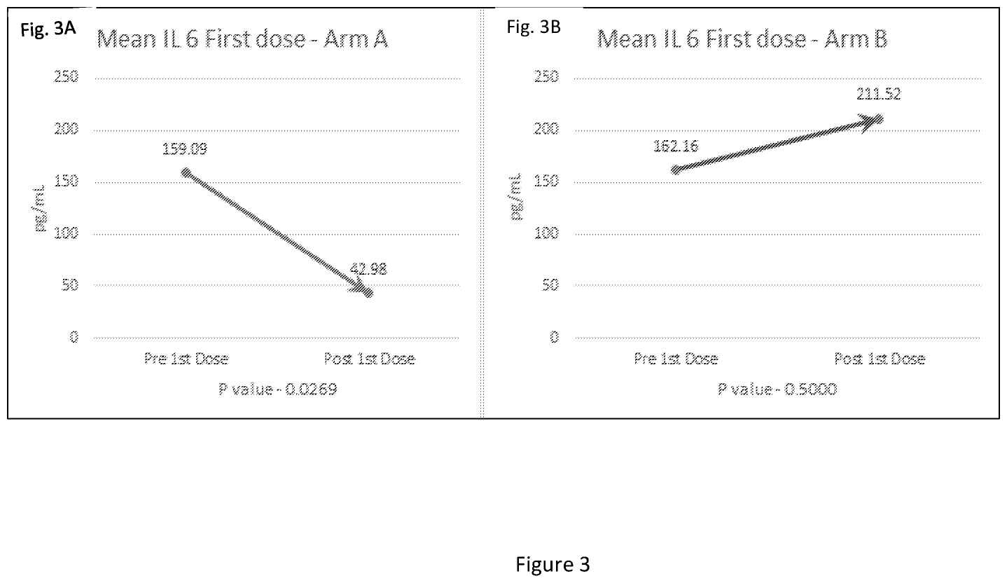

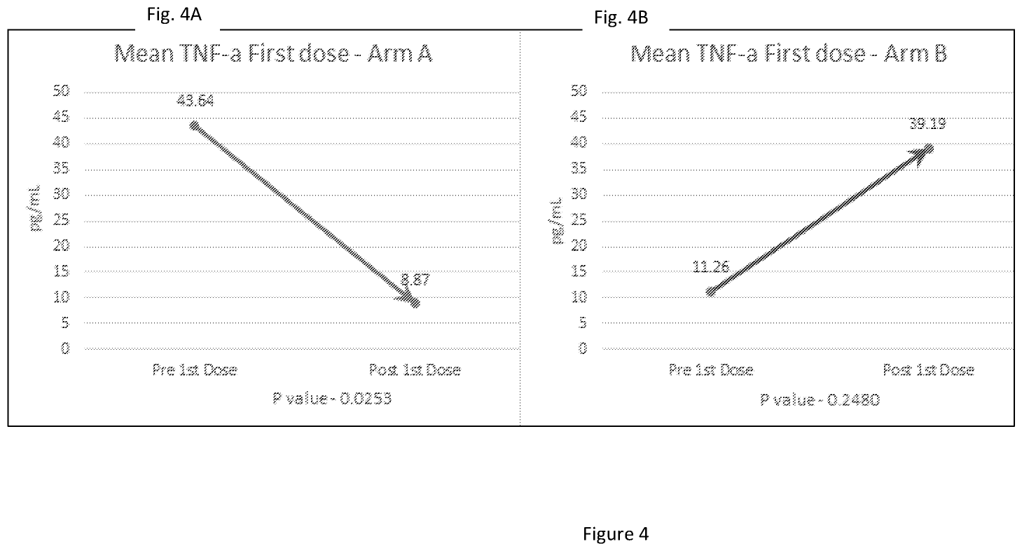

Itolizumab treatment had a noticeable improvement on patient's survival through reduction in 1-month mortality rate. A statistically significant difference (p=0.0098) in the 1-month mortality rate was observed between the treatment arm A and B. All patients dosed with Itolizumab consistently demonstrated improvement in the pulmonary function parameters:
Significantly stable/improved oxygen saturation (SpO₂) and PaO₂ without increasing FiO₂.
The O₂ requirement reduced post dosing.
The PaO₂/FiO₂ ratio improved consistently in all patients.
Overall, a higher proportion of patients in Arm A had stable/improved SpO₂ without increasing FiO₂. Overall, a higher proportion of patients in Arm A had stable PaO₂ without increasing FiO₂ in all the post-baseline assessment visits. A statistically significant difference from Day 21 onwards was observed between both treatment arms for both parameters.
PFR (PaO₂/FiO₂ ratio) gradually increase over time in Arm A and B. Mean PFR was 350.25 in Arm A and 398.33 in Arm B over 21 days.
Biomarkers such as IL-6, IL-17A and TNF-α demonstrated decrease post Itolizumab treatment. Patients demonstrated greater reduction in mean change from baseline levels compared to control arm.
   IL-6 is a pro-inflammatory cytokine. As seen in FIG. 3 mean baseline value of IL-6 was comparable in both arms; 159.1 pg/mL in Arm A and 162.2 pg/mL in Arm B. A significant decline (p=0.0269) in mean IL-6 levels post first infusion was seen in Arm A (43 pg/mL) compared to Arm B (212 pg/mL). p-value estimated using Wilcoxon matched-pairs signed rank test.
   TNF-α is a pro-inflammatory cytokine. As seen in FIG. 4 mean baseline value of TNF a was higher in Arm A (44 pg/mL) than in Arm B (11 pg/mL). A significant decline (p=0.0253) in mean TNF-α levels post first infusion was seen in Arm A (9 pg/mL) compared to an increase in Arm B (39 pg/mL). Pre second dose TNF-α levels were 68 pg/mL and 108 pg/mL in Arm A and B, respectively. Post second dose, a decrease in TNF-α levels was also seen in Arm A (50 pg/mL) versus an increase in Arm B (185 pg/mL). p-value estimated using Wilcoxon matched-pairs signed rank test
   IL-17A is a pro-inflammatory cytokine. Mean baseline value of IL-17A was comparable in both arms; 10.36 pg/mL in Arm A and 9.83 pg/mL in Arm B. A notable decline in mean IL-17A levels post first infusion was seen in Arm A (6.75 pg/mL) unlike in Arm B, where there was an increase (14.75 pg/mL). A slight decrease in IL-17A levels was also seen post second dose in Arm B versus an increase in Arm B.
Other important markers of organ dysfunction and coagulopathy like LDH (Lactate Dehydrogenase) and D-Dimer also demonstrated greater mean reduction in in Arm A compared to control arm.

Primary endpoint of mortality was statistically highly significant in favour of Itolizumab arm; p value=0.0098. Itolizumab consistently demonstrated improvement in the pulmonary function parameters and decrease in inflammatory biomarker levels. Itolizumab was safe in COVID-19 patients. Infusion reactions were manageable with slowing infusion rate. Itolizumab effectively controlled hyper-activation of the immune system in response to COVID-19 virus and reduced morbidity and mortality related to cytokine storm.

Overall, Itolizumab treatment was well tolerated when administered to COVID-19 patients along with the BSC (Best supportive care) with no new safety concerns emerging due to the therapy.

### Example 2. The Use of Itolizumab (SEQ ID Nos: 4 and 6) Reduced the Mortality Rate in Severely and Critically Ill Patients

48 patients diagnosed with SARS-CoV-2 or with high clinical suspicion of COVID-19 pneumonia and clinical, radiological or laboratory evidence of cytokine release syndrome were administered a 200 mg dose of the monoclonal antibody Itolizumab intravenously. Out of these, 22 patients (14 critically and 8 severely ill) received a second dose of the antibody after 72 hours and 3 critically ill patients received a third dose since they had persistent signs of respiratory insufficiency or macrophage activation syndrome. To evaluate the impact of the use of the product in research, every COVID-19 positive patient with information on recovery or death reported to the Ministry of Public Health with a history of at least one comorbidity considered a risk factor for severe/fatal COVID-19 outcomes (hypertension, ischemic heart disease, diabetes mellitus, cancer, chronic kidney disease, obesity, malnutrition or COPD) or that had been admitted to an ICU in the country and that had not been included in any of the ongoing clinical trials for COVID-19 was selected as control. For the statistical processing of the data, the χ² test was employed.

As can be observed in Table 3 the mortality rate of Itolizumab-treated patients was 15% lower than that of the control group. Furthermore, the mortality rate of severely ill patients treated with Itolizumab was significantly lower as compared to that of severely ill patients of the control group.

TABLE 3
Mortality of severely or critically ill patients

| Classification | | | | |
|---|---|---|---|---|
| of Patients | | | | |
| according to | | | | |
| clinical | | Control | | P Value X² |
| criterium | | Group | Itolizumab | Test |
| Critically ill | Quantity of patients | 25 | 26 | |
| | Deceased | 22 | 21 | |
| | Mortality rate (%) | 88% | 80.8% | 0.48 |
| Severely ill | Quantity of patients | 26 | 22 | |
| | Deceased | 12 | 4 | |
| | Mortality rate (%) | 46.2% | 18.2% | 0.04 |
| Severely and | Total mortality rate | 66.6% | 52.08% | — |
| critically ill | | | | |
| patients | | | | |

### Example 3. The Use of Itolizumab Reduced the Mortality of Moderately Ill Patients with High Risk of Becoming Severely or Critically Ill

A sample of 14 moderately ill patients, with 2 or more comorbidities that predict mortality, were administered a 200 mg [dosage of 3.2 mg/kg of body weight] of the monoclonal antibody Itolizumab (SEQ ID Nos: 4 and 6) intravenously. The patients that were not diagnosed while being in an intensive care unit and that had 2 or more comorbidities were selected as untreated controls. For the statistical processing of the data, the test of χ² was used.

As can be seen in Table 4, the mortality rate of patients at high-risk of becoming severely or critically ill patients decreased by 27%.

TABLE 4
Mortality rate of moderately ill patients.

| | Control group | Itolizumab | P Value χ² Test |
|---|---|---|---|
| Total number of patients | 52 | 14 | 0.07 |
| Admitted to ICU | 29 | 4 | |
| Frequency (%) | 55.8 | 28.6 | |

### Example 4. Itolizumab Treatment Reduced the IL-6 Serum Concentrations in Critically and Severely COVID-19 Positive Ill Patients and Stabilized Such Levels in Moderately Ill Patients

In 21 COVID-19 positive patients treated with the monoclonal antibody Itolizumab (SEQ ID Nos: 4 and 6), the IL-6 serum concentrations were determined by ELISA (Quantikine), before starting treatment and 48 hours after administration. The patients were classified as: moderately ill: n=12; severely ill: n=4 and critically ill: n=5.

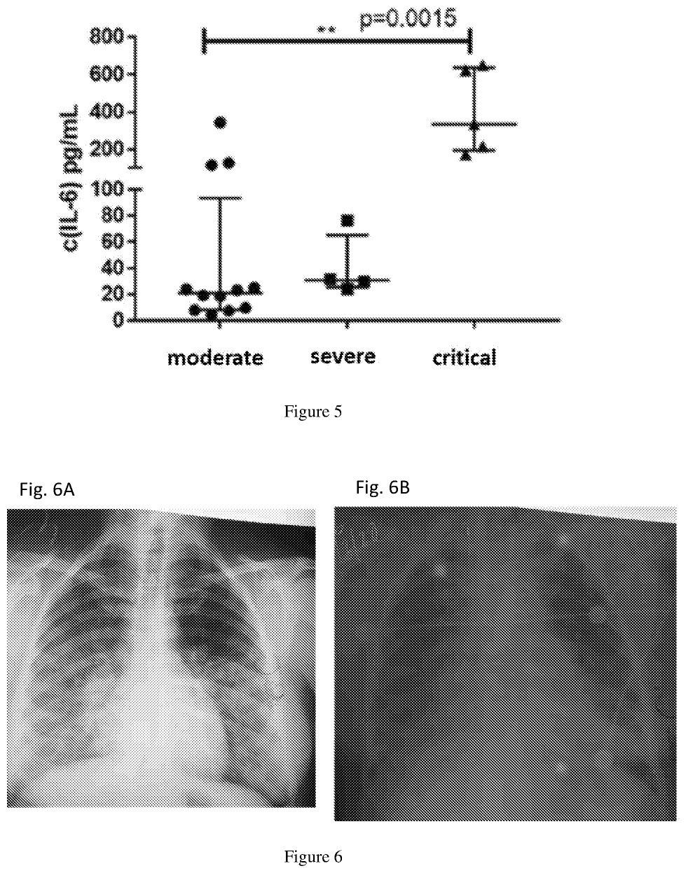

As can be seen in FIG. 5, before starting treatment with Itolizumab, the IL-6 levels of the patients increased with disease progression. The serum concentrations of critically ill patients were significantly higher than those of moderately ill patients (Kruskal-Wallis test; p=0.0015). Baseline IL-6 levels were related to the severity of the disease by applying a ROC curve, the selected cut-off value of the IL-6 serum concentration was 27.4 pg/ml.

In a group of 16 patients who received the Itolizumab, the IL-6 serum concentrations before the first administration and 48 hours later were evaluated. Table 5 shows the variation in IL-6 values at 48 hours as a function of the established cut-off value.

All patients with circulating levels of IL-6 greater than 27.4 pg/ml decreased their values with one dose of Itolizumab, measured between 24 and 48 hours after administration. The magnitude of the change in IL-6 concentration among patients with concentrations above the cut-off had a median reduction of 50.9 pg/ml. However, the median change in IL-6 concentration among patients with baseline levels less than 27.4 pg/ml was 1.5 pg/ml.

TABLE 5
Variation in IL-6 values at 48 hours as a function of
the threshold that discriminates disease severity

| | | IL-6 ini > 27.4 | IL-6 ini ≤ 27.4 |
|---|---|---|---|
| Before | Number of patients | 9 | 7 |
| Treatment | Median ± ri | 127.4 ± 285.2 | 19.1 ± 16.5 |
| | (min; max) | (30.0; 623.1) | (4.4; 24.9) |
| 48 h after | Median ± ri | 78.8 ± 149.0 | 24.2 ± 75.8 |
| treatment | (min; max) | (11.5; 372.9) | (5.9; 156.4) |
| 48 h - | Median ± ri | −50.9 ± 152.0 | 1.5 ± 59.3 |
| initial value | (min; max) | (−262.8; −10.2) | (−4.8; 137.3) |
| P (Wilconxon) | | 0.008 | 0.116 |

### Example 5. Clinical Improvement of a Severely Ill Patient, with a Cytokine Storm Caused by a Respiratory Infection of Bacterial Origin, Treated with Itolizumab

A female patient with a diagnosis of extra hospitality bronchopneumonia (FIG. 6A) with a guarded prognosis and a history of being an inveterate smoker suffering from Bronchial Asthma received a dose of about 200 mg intravenously of mAb Itolizumab (SEQ ID Nos: 4 and 6. Her vital parameters at the time of admission were: temperature 38.2° C., respiratory rate 120 and heart rate 89, blood pressure 90/60, SatO₂ 98, PO₂ 116, PCO₂ 88.6, creatinine 50, hematocrit 0.32, leukogram 16.5×10⁹, lymphocytes 0.24 and monocytes 0.02, platelets 350×10⁹, parameters that indicated the patient was experiencing a cytokine storm. In addition to the mAb, she received concomitant treatment with Meropenem and Vancomycin, Gentamicin, Oseltamivir, Omeprazole, Flaxiheparin, Morphine, Midazolam, vitamin therapy, Chlorhexidine oral cavity wash at 0.1%.

Radiological improvement was observed at 48 hours as well as significant improvement in vital parameters (FIG. 6B). The computerized axial tomography performed ten days after the administration of the antibody showed no signs of interstitial pneumonia, only calcified nodules in the base of the lungs, which are commonly found in inveterate smokers such as this patient. Furthermore, no adverse events associated with the administration of the antibody were reported. There were no subsequent infections. Fourteen days after admission, the patient was medically discharged.

### Example 6. Itolizumab Treatment Showed a High Rate of Recovery in COVID-19 Suspected Patients not Confirmed by PCR

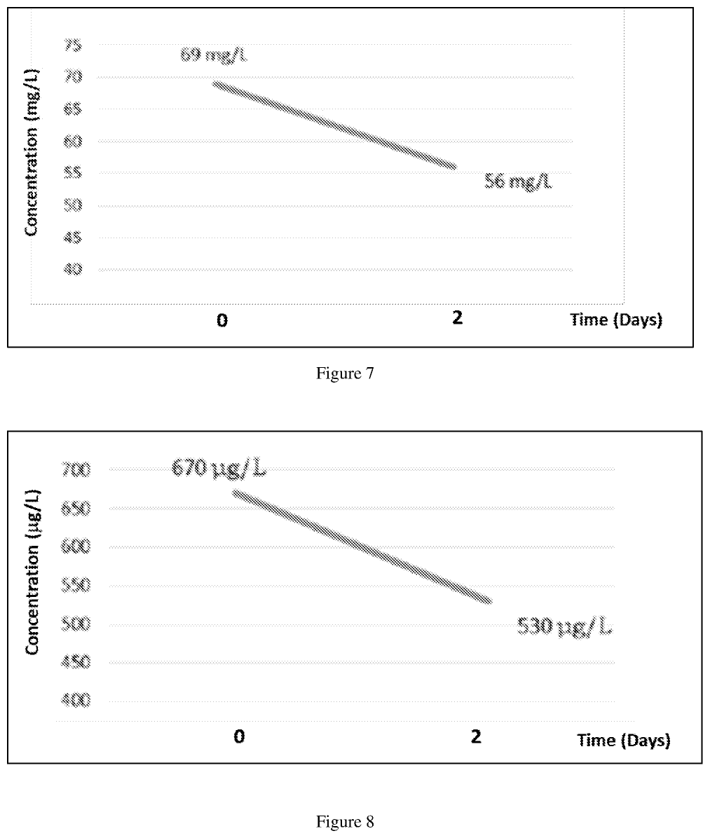

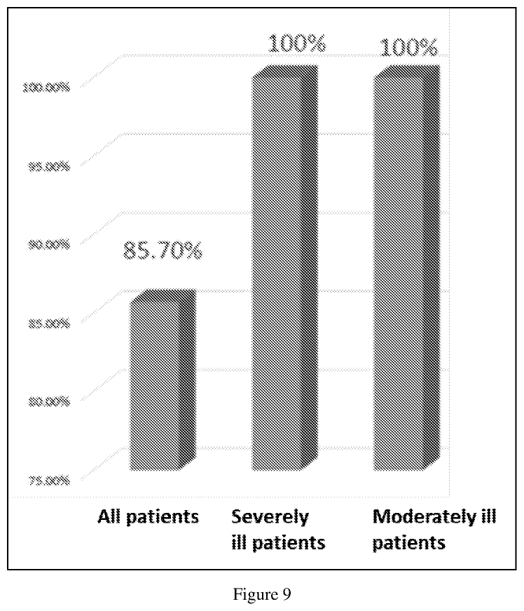

A total of 22 patients admitted at Roberto Rodriguez Hospital (Moron, Ciego de Avila) due to a clinical suspicion of COVID-19 pneumonia (not confirmed by PCR), received an intravenously 200 mg dose of Itolizumab (SEQ ID Nos: 4 and 6) and two doses of this antibody in one of the cases. The patients when admitted at the hospital were in critical, severe or moderate condition with a high risk of aggravation. The concentration of the inflammatory parameters was determined before the administration of the antibody and 48 hours later. The media values of C-reactive protein concentration (FIG. 7) and ferritin concentration (FIG. 8), decreased 48 hours after treatment, evidencing that Itolizumab therapy caused a reduction in markers associated with severe COVID disease and/or cytokine storm. FIG. 9 shows that despite the high levels of inflammation observed before the administration of the antibody there was a recovery in all moderate and severe patients and in 86% of all treated patients.

### Example 7. Treatment of Patients with COVID-19 Requiring Hospitalization

The trial included male or female adults above 18 years with a confirmed virological diagnosis of SARS-CoV-2 infection with RT-PCR and requirement of hospitalization due to clinical worsening of COVID-19 infection with an oxygen saturation at rest in ambient air ≤94%. Patients with moderate to severe ARDS, as defined by PaO₂/FiO₂ ratio of <200, or more than 25% deterioration from the immediate previous value and patients with baseline serum ferritin level ≥400 ng/mL or IL-6 levels greater than 4 times the upper limit of normal (ULN) were included.

The first dose of Itolizumab (SEQ ID NOs. 4 and 6) was administered at 1.6 mg/kg. An in-vitro CD6 receptor occupancy was evaluated and the 1.6 mg/kg dose showed a 99% receptor occupancy. In some patients, an additional dose of 0.8 mg/kg was administered after 1 week, if required. As the patients experienced different degrees of host inflammatory response, subsequent weekly doses were not necessary in all patients. The decision was left to the investigator's discretion based on the clinical condition and markers of inflammation. Up to four weekly doses were allowed in the study.

### Example 8. Treatment of Patients with COVID-19 Prior to Symptoms of Cytokine Storm

The first dose of Itolizumab (SEQ ID NOs. 4 and 6) was administered at 1.6 mg/kg. This loading dose of 1.6 mg/kg was chosen as it is the approved dose in patients of chronic plaque psoriasis and doses up to 1.6 mg/kg have been administered as i.v. infusion in several phase 2 and 3 clinical trials, without any evidence of dose-limiting toxicities. As Itolizumab is an anti-CD6 antibody, an in-vitro CD6 receptor occupancy was evaluated and as 1.6 mg/kg dose showed a 99% receptor occupancy, it was chosen as the first dose. In some patients, an additional dose of 0.8 mg/kg was administered after 1 week, if required. As the patients experienced different degrees of host inflammatory response, subsequent weekly doses were not necessary in all patients. The decision was left to the investigator's discretion based on the clinical condition and markers of inflammation. Up to four weekly doses were allowed in the study.

### Example 9. Treatment of Hospitalized COVID-19 Patients with Itolizumab

Twenty subjects with COVID-19 receiving standard of care therapy are administered an initial 1.6 mg/kg intravenous infusion of Itolizumab (SEQ ID NOs. 4 and 6). In subjects with no or mild adverse symptoms from the initial dose and who continue to experience symptoms of COVID-19, they are administered an additional 1.6 mg/kg every two weeks. Six subjects received one dose at 1.6 mg/kg Itolizumab; seven subjects received two doses at 1.6 mg/kg with two weeks between treatments; three subjects received one 1.6 mg/kg dose Itolizumab and after a week they received 0.8 mg/kg and after another week they received a last treatment of 0.8 mg/kg Itolizumab; four subjects received one 1.6 mg/kg dose Itolizumab and for three weeks following, with a week elapsing between each dose, they received three additional doses at 0.8 mg/kg for a total of four treatments.

### Example 10. Treatment of COVID-19 Patients with Itolizumab or Placebo in Addition to Standard of Care Therapy

Patients with COVID-19 are split into two groups, one receiving standard of care therapy plus placebo and one receiving an initial 1.6 mg/kg (based on ideal body weight) intravenous infusion of Itolizumab in addition to the standard of care therapy. On Day 8 patients in the treatment group receive an additional 0.8 mg/kg if they have:—a) not been discharged from the hospital; b) not recovered in the hospital; c) not had anaphylaxis with the first dose; d) not have ALC 0.5; and d) not be diagnosed with active tuberculosis

1. Use of a non-depleting anti-CD6 antibody that specifically binds to domain 1 of CD6 for the treatment of infectious disease that leads to a cytokine storm or a cytokine release syndrome wherein the non-depleting anti-CD6 antibody comprising of amino acids in SEQ ID Nos: 1 and 2 or a non-depleting anti-CD6 antibody that specifically binds to domain 1 of CD6 having 98% identity to SEQ ID Nos: 1 and 2.

2. The use of a non-depleting anti-CD6 monoclonal antibody according to claim 1 wherein the infectious diseases are caused by the pathogens selected from the group comprising of:—cytomegalovirus, coronavirus, Epstein-Barr virus, group A streptococci, avian influenza virus, influenza virus, smallpox virus, dengue virus, pneumococci.

3. Use of a non-depleting anti-CD6 antibody according to claim 1 that specifically binds to domain 1 of CD6 for the treatment of infectious disease caused by the coronavirus that lead to a cytokine storm or a cytokine release syndrome.

4. The use according to claim 3, wherein the coronavirus is SARS, MERS-CoV or COVID-19 coronavirus and variants thereof.

5. Use of a non-depleting anti-CD6 antibody according to claim 1 that specifically binds to domain 1 of CD6 for the treatment of the infectious diseases caused by the bacterial agent selected from group A streptococci and pneumococci.

6. The use according to claim 1, where the non-depleting anti-CD6 antibody that specifically binds to domain 1 of CD6 and having 98% identity to SEQ ID Nos: 1 and 2 comprises of SEQ ID NO: 1 and SEQ ID NO: 3.

7. Use of an anti-CD6 antibody that specifically binds to domain 1 of CD6 for the treatment of COVID-19 and variants thereof wherein the anti-CD6 antibody reduces expression of cytokines and comprises amino acids in SEQ ID Nos: 4 and 5 or an anti-CD6 antibody that specifically binds to domain 1 of CD6 having 98% identity to SEQ ID Nos: 4 and 5.

8. The use according to claim 7, where the anti-CD6 antibody that specifically binds to domain 1 of CD6 and having 98% identity to SEQ ID Nos: 4 and 5 comprises of SEQ ID NO: 4 and SEQ ID NO: 6.

9. The use according to prior claims wherein the anti-CD6 antibody is in an effective amount of between 0.5 mg/kg of body weight and 10 mg/kg of body weight.

10. A method of treating a coronavirus-infected subject comprising treatment with a therapeutically effective amount of an anti-CD6 antibody comprising amino acid sequences as set forth in SEQ ID NOs: 4 and 5 or an amino acid sequence having at least 98% identity thereto comprising SEQ ID Nos: 4 and 6, wherein the anti-CD6 antibody reduces high expression levels of cytokines formed during a cytokine storm.

11. The method according to claim 10, wherein the coronavirus is SARS, MERS-CoV or COVID-19 coronavirus.

12. A method of treating a bacteria-infected subject comprising treatment with a therapeutically effective amount of an anti-CD6 antibody comprising amino acid sequences as set forth in SEQ ID NOs: 4 and 5 or an amino acid sequence having at least 98% identity thereto comprising SEQ ID Nos: 4 and 6, wherein the anti-CD6 antibody reduces high expression levels of cytokines formed during a cytokine storm.

13. The method of treating bacterial-infection according to claim 12 wherein the infectious diseases are caused by the pathogens selected from the group comprising of: group A streptococci and pneumococci.

14. The method of claim 10-13, wherein the therapeutically effective amount of an anti-CD6 antibody is between 0.5 mg/kg of body weight and 10 mg/kg of body weight.

15. The method of claim 14, wherein the anti-CD6 antibody is administered at least twice to the subject, with time elapsed between two consecutive administrations is between 24 and 96 hours.

16. A composition for alleviating the cytopathic destructive effects of COVID-19 infection in a human patient comprising a therapeutically effective amount of an anti-CD6 antibody comprising SEQ ID NO: 4 and SEQ ID NO: 5 or SEQ ID NO: 6 further comprising one or more one active agent selected from Reverse transcriptase (RT) inhibitors, Protease inhibitors, Fusion inhibitors, Cytosorp, IL6 receptor antagonists or JAK inhibitors.

17. The composition of claim 16, wherein the therapeutically effective amount of an anti-CD6 antibody is between 0.5 mg/kg of body weight and 10 mg/kg of body weight.

18. The composition of claim 16, wherein the anti-CD6 antibody is administered at least twice to the subject, with time elapsed between two consecutive administrations is between 24 and 96 hours.

19. A kit for treating a subject against COVID-19 and variants thereof wherein the kit comprises a therapeutically effective amount of an anti-CD6 antibody consisting of an amino acid sequence of SEQ ID NOs 4 and 5 or an amino acid sequence having 98% identity thereto consisting of SEQ ID Nos: 4 and 6.

20. Use of an anti-CD6 antibody in the manufacture of a medicament for the treatment or prevention of cytokine storm exhibited by infection of a coronavirus or bacterial agent such as group A streptococci and pneumococci in a mammal, the anti-CD6 antibody comprising an amino acid sequence of SEQ ID NO: 4 and SEQ ID NO: 5 or 6.

21. The use of claim 20, wherein the coronavirus is SARS, MERS-CoV or COVID-19 coronavirus.

22. A method of treating a coronavirus-infected subject comprising treatment with a therapeutically effective amount of an anti-CD6 antibody comprising amino acid sequences as set forth in SEQ ID NOs: 4 and 6, wherein the concentrations of C-reactive protein and ferritin are reduced within 48 hours after initial treatment.

23. The use or method according to any one of claims 1-22, wherein the anti-CD6 antibody is a humanized IgG1 monoclonal antibody.

24. The use or method according to claim 23, wherein the anti-CD6 antibody is Itolizumab.


SEQUENCE LISTING (6 sequences)

SEQ ID NO: 1 - protein, 119 aa, Artificial Sequence
  Synthetic Construct
EVQLVESGGGLVKPGGSLKLSCAASGFKFSRYAMSWVRQAPGKRLEWVATISSGGSYIYYPDSVKGRFTISRDNVKNTLYLQMSSLRSEDTAMYYCARRDYDLDYFDSWGQGTLVTVSS

SEQ ID NO: 2 - protein, 107 aa, Artificial Sequence
  Synthetic construct
DIQMTQSPSSLSASVGDRVTITCKASRDIRSYLTWYQQKPGKAPKTLIYYATSLADGVPSRFSGSGSGQDYSLTISSLESDDTATYYCLQHGESPFTLGSGTKLEIK

SEQ ID NO: 3 - protein, 109 aa, Artificial Sequence
  Synthetic construct
DIQMTQSPSSLSASVGDRVTITCKASRDIRSYLTWYQQKPGKAPKTLIYYATSLADGVPSRFSGSGSGQDYSLTISSLESDDTATYYCLQHGESPFTFGSGTKLEIKRA

SEQ ID NO: 4 - protein, 449 aa, Artificial Sequence
  Synthetic Construct
EVQLVESGGGLVKPGGSLKLSCAASGFKFSRYAMSWVRQAPGKRLEWVATISSGGSYIYYPDSVKGRFTISRDNVKNTLYLQMSSLRSEDTAMYYCARRDYDLDYFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 214 aa, Artificial Sequence
  Synthetic Construct
DIQMTQSPSSLSASVGDRVTITCKASRDIRSYLTWYQQKPGKAPKTLIYYATSLADGVPSRFSGSGSGQDYSLTISSLESDDTATYYCLQHGESPFTLGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 216 aa, Artificial Sequence
  Synthetic Construct
DIQMTQSPSSLSASVGDRVTITCKASRDIRSYLTWYQQKPGKAPKTLIYYATSLADGVPSRFSGSGSGQDYSLTISSLESDDTATYYCLQHGESPFTFGSGTKLEIKRARTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC